(19) United States
(12) Patent Application Publication
(10) Pub. No.: US 2013/0123222 A1
(43) Pub. Date: May 16, 2013

(54) Synergistic Anti-Cancer Activity of SR16388 with Anti-Mitotic Drugs

(71) Applicant: SRI INTERNATIONAL, INC., Menlo Park, CA (US)

(72) Inventors: Wei Zhou, Fremont, CA (US); Lidia Sambucetti, Redwood City, CA (US)

(73) Assignee: SRI INTERNATIONAL, INC., Menlo Park, CA (US)

(21) Appl. No.: 13/674,071

(22) Filed: Nov. 11, 2012

Related U.S. Application Data
(60) Provisional application No. 61/558,079, filed on Nov. 10, 2011.

(51) Int. Cl. A61K 31/57; A61K 31/475; A61K 31/337

(52) U.S. Cl. CPC A61K 31/57; A61K 31/337; A61K 31/475
USPC 514/171

(57) ABSTRACT

The invention provides methods and compositions for inhibiting tumor growth in a mammal. The methods comprise administering to the mammal a synergistic combination of (E)-3-hydroxy-21-[2′-(N,N-dimethylamino)ethoxy]-19-norpregna-1,3,5(10),17(20)-tetraene and a microtubulin inhibitor like paclitaxel or vincristine. The combination of the compounds more than additively inhibits growth of tumor cells.

### CROSS-REFERENCE TO RELATED APPLICATIONS

This application claims priority to U.S. Ser. No. 61/558,079, filed Nov. 10, 2011, the disclosure of which is incorporated herein by reference in its entirety.

### INTRODUCTION

Synergistic effects of pharmaceutical compounds can be used to design combination therapy regimens that are more effective and have lower toxicity compared with regimens using the individual compounds.

Due to their ability to disrupt cell division, mitotic inhibitors have been used previously as anti-cancer medications. Cancer cells grow and spread through unregulated mitotic division, thus allowing mitotic inhibitors to have anti-cancer effects. Improved cancer treatments focusing on the use of mitotic inhibitors are continuously being sought in the medicinal arts.
Relevant Art: U.S. Pat. No. 6,054,446, U.S. Pat. No. 8,268,807, and U.S. Pat. No. 6,281,205; Chao et al. Angiogenesis 2011 March; 14(1):1-16. Epub 2010 Nov. 21; Duellman et al., Biochem Pharmacol. 2010 Sep. 15; 80(6):819-26. Epub 2010 May 31.

### SUMMARY OF THE INVENTION

The invention provides synergistic combinations of SR16388 and a microtubulin inhibitor, methods of using the combinations to inhibit tumor or cancer cell growth, and methods of making compositions comprising the combinations.

In one aspect the invention provides a method for inhibiting tumor growth in a mammal comprising administering to the mammal a synergistic combination of SR16388 and a microtubulin inhibitor. In particular embodiments:

the combination more than additively inhibits growth of ovarian, colon, breast, prostate, lung, or myeloma tumor cells;

the combination is administered in a single composition;

one or both of the SR16388 and inhibitor is administered in an amount subtherapeutic if administered alone;

one or both of the SR16388 and inhibitor is administered in an amount less than its IC₅₀ for said cells;

the combination is administered in an amount less than its IC₅₀ for said cells;

the inhibitor is selected from the group consisting of paclitaxel, docetaxel, vinblastine, vinorelbine, and vincristine, or combinations thereof, particularly, paclitaxel or vincristine;

the SR16388:inhibitor ratio is between 90:10 and 60:40;

combination is synergistic for a plurality of cell lines selected from NCIH460, SKMEL28, MCF7, UACC257, SNB19, COLO205, UACC62, HS578T, SKMEL2, HL60, SF268, T47D, NCIH226, CAKI1, HT29, 786O, SKOV3, MOLT4, NCIH322M, SF539, MDAMB231, HCT15, HCT116, SKMEL5, TK10, SF295, CCRFCEM, MDAMB435, SN12C, MALME, OVCAR8, NCIH522, SNB75, SW620, A549, A498, HCC2998, HOP92, IGROV1, ACHN, LOXIMVI, UO31, U251, KM12, MDAMB468, K562, OVCAR3, BT549, NCIH23, OVCAR5, RPMI8226, ADRRES, DU145, PC3, HOP62, EKVX, and SR;

the combination exhibits greater synergy compared with a composition comprising SR16388 and an anti-cancer drug selected from SN38, Dasatinib, Romidepsin, Actinomycin, Tamoxifen, Rapamycin, Lapatinib, Sunitinib, Imatinib, Carboplatin, Everolimus, Nilotinib, Azacytidine, Capecitabine, Celecoxib, Cladribine, Clofarabine, Cytarabine, Floxuridine, FluroaraA, Gemcitabine, Letrozole, Nelarabine, Temozolomide, Thiotepa, Tretinoin, and Vorinostat.

The inventions encompasses combinations of particular embodiments, such as wherein:

the inhibitor is paclitaxel, the combination is administered in a single composition, and the SR16388:inhibitor ratio is between 85:15 and 65:35;

the inhibitor is paclitaxel or vincristine, the combination is administered in a single composition, and the inhibitor is administered in an amount subtherapeutic if administered alone; or

the inhibitor is paclitaxel or vincristine, the combination is administered in a single composition, and the tumor is an ovarian, colon, breast, prostate, lung or myeloma tumor.

In particular embodiments the method further comprises the step of detecting a resultant inhibition of tumor growth.

In another aspect the invention provides a pharmaceutical composition comprising a synergistic, with respect to tumor growth inhibition, combination SR16388 and a microtubulin inhibitor, particularly wherein the composition provides a synergistic inhibition of ovarian, colon, breast, prostate, lung or myeloma tumor growth.

In another aspect, there is provided a pharmaceutical composition comprising SR16388 and a microtubulin inhibitor selected from paclitaxel and vincristine, wherein the pharmaceutical composition provides a synergistic effect in the treatment of a cancer selected from ovarian, colon, and myeloma cancer.

In further embodiments:

the composition comprises the microtubulin inhibitor in an amount greater than or equal to 3.0E-7M.

the microtubulin inhibitor is present in an amount greater than or equal to 3.0E-7M and that is less than the average IC50 for the microtubulin inhibitor as determined in an equivalent single compound test, wherein the single compound test is against cancer cells selected from colon, ovarian, breast, prostate, and lung cancer.

the SR16388 is present in an amount greater than or equal to 2.0E-7M.

The invention specifically provides all combinations of the recited aspects, as if each had been laboriously individually set forth.

### DETAILED DESCRIPTION OF PARTICULAR EMBODIMENTS

The methods and compositions, including combinations, use the compound (E)-3-hydroxy-21-[2′-(N,N-dimethylamino)ethoxy]-19-norpregna-1,3,5(10),17(20)-tetraene. For convenience, the compound will alternatively be referred to herein as SR16388. SR16388 has the structure shown below.

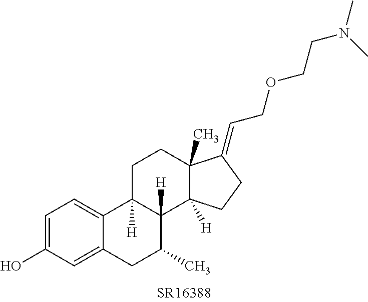

SR16388 can be used in pharmaceutically acceptable alternative forms, such as pharmaceutically acceptable salts, esters, ethers, prodrugs (e.g. sulfamates, phosphates), and the like. Unless otherwise specified, all references herein to “SR16388” are intended to include such alternative forms. Pharamceutically acceptable and pharmaceutically active combinations of such forms, such as salts of prodrugs, are possible and within the scope of the disclosure as well. Salts, esters, amides, prodrugs, active metabolites, analogs, and other derivatives of the active agents may be prepared using standard procedures known to those skilled in the art of synthetic organic chemistry and described, for example, by J. March, Advanced Organic Chemistry: Reactions, Mechanisms and Structure, 5th Ed. (New York: Wiley-Interscience, 2001). Furthermore, where appropriate, functional groups on the compounds of the disclosure may be protected from undesired reactions during preparation or administration using protecting group chemistry. Suitable protecting groups are described, for example, in Green, Protective Groups in Organic Synthesis, 3rd Ed. (New York: Wiley-Interscience, 1999).

According to the invention, synergism is found for SR16388 with microtubulin inhibitors (anti-mitotic compounds), particularly within the concentration ranges and ratios described. As observed in the Examples provided herein, synergism is not found for S16388 with other anti-cancer agents (i.e., anti-cancer agents not functioning as microtubulin inhibitors), such as anti-proliferation agents, kinase inhibitors, aromatase inhibitors, differentiation inducers, etc.

By “synergism” or “synergy” is meant that the effect with the combination of agents is greater than the additive effect of the agents alone.

In embodiments, synergism is determined by measuring IC₅₀ values (such as by the method described herein or others).

In embodiments, the microtubulin inhibitor is paclitaxel, docetaxel, vinblastine, vinorelbine, or vincristine, or combinations thereof; preferably paclitaxel or vincristine; more preferably paclitaxel.

In embodiments, the composition comprises the microtubulin inhibitor (and/or SR16388) in an amount that is less than the average IC₅₀ for the inhibitor (and/or SR16388) as determined in an equivalent single compound test against. Such a single compound test can be conducted using the procedures described herein, or another suitable procedure. For example, the test can be against cancer cells selected from colon, ovarian, breast, prostate, and lung cancer. The conditions can be, for example, a 24-hour exposure of the cancer cells to a composition comprising the compound. For example, the combined concentration of SR16388 and the inhibitor is or is at least 10, 20, 30, 40, 50% less than the average IC₅₀ for SR16388 as determined in an equivalent single compound test.

In embodiments, more of SR16388 is present in the composition compared with the microtubulin inhibitor (i.e., the ration is greater than 50:50). For example, the ratio of SR16388:microtubulin inhibitor is between 90:10 and 60:40, or between 85:15 and 65:35. For example, the ratio is greater than 55:45, or 60:40, or 65:35, or 70:30, or 75:25, or 80:20. In embodiments, the amount of SR16388 in the composition is greater than 55, 60, 65, 70, 75, 80, or 85%.

In embodiments, the composition is synergistic for at least 1, 2, 3, 4, 5, 6, 7, 8, 9, 10, 20, 30, 40 or 50 cell lines selected from NCIH460, SKMEL28, MCF7, UACC257, SNB19, COLO205, UACC62, HS578T, SKMEL2, HL60, SF268, T47D, NCIH226, CAKI1, HT29, 786O, SKOV3, MOLT4, NCIH322M, SF539, MDAMB231, HCT15, HCT116, SKMEL5, TK10, SF295, CCRFCEM, MDAMB435, SN12C, MALME, OVCAR8, NCIH522, SNB75, SW620, A549, A498, HCC2998, HOP92, IGROV1, ACHN, LOXIMVI, UO31, U251, KM12, MDAMB468, K562, OVCAR3, BT549, NCIH23, OVCAR5, RPMI8226, ADRRES, DU145, PC3, HOP62, EKVX, and SR.

In embodiments, the composition exhibits greater synergy compared with a composition comprising SR16388 and at least one or a plurality of anti-cancer drugs that are not microtubulin inhibitors or not anti-mitotic. In embodiments, the composition exhibits greater synergy compared with a composition comprising SR16388 and an anti-cancer drug selected from SN38, Dasatinib Romidepsin, Actinomycin, Tamoxifen, Rapamycin, Lapatinib, Sunitinib, Imatinib, Carboplatin, Everolimus, Nilotinib, Azacytidine, Capecitabine, Celecoxib, Cladribine, Clofarabine, Cytarabine, Floxuridine, FluroaraA, Gemcitabine, Letrozole, Nelarabine, Temozolomide, Thiotepa, Tretinoin, and Vorinostat.

In embodiments, the tumor cells to be treated are selected from ovarian, colon, breast, prostate, lung, and myeloma cancer cells, particularly present in a mammal, particularly a human. In embodiments, the growth of the tumor or tumor cells in the mammal is suppressed, e.g. slowed by at least 50, 70, 90 or 100%. In embodiments, the tumor is a solid tumor, or a metastatic tumor.

In other aspects the invention provides:

a method for reducing the number of tumor cells in a mammal comprising: administering to the mammal a composition comprising a synergistic combination of SR16388 and a microtubulin inhibitor, wherein the administered composition is effective to substantially reduce the number of tumor cells to a level more than additive when compared to administration of SR16388 and the inhibitor alone;

a method for synergistically enhancing the effectiveness of a microtubulin inhibitor comprising administering an effective dose of SR16388 in conjunction with the inhibitor, particularly wherein the inhibitor is paclitaxel or vincristine; or

a method for suppressing tumor growth in a mammal comprising: administering to the mammal a synergistic combination of SR16388 and at least one microtubulin inhibitor, particularly paclitaxel or vincristine, in a combined dosage effective to substantially reduce the targeted tumor cell population to a level more than additive when compared to administration of SR16388 and the inhibitor alone, wherein said tumor growth in said mammal is suppressed.

In an aspect, there is provided a pharmaceutical composition comprising SR16388 and a microtubulin inhibitor, such as paclitaxel or vincristine, wherein the pharmaceutical composition provides a synergistic effect in the treatment of a cancer selected from ovarian, colon, breast, prostate, lung, and myeloma cancer.

In embodiments, the composition may further comprise one or more additives such as pharmaceutically acceptable excipients, solvents, carriers, colorants, pH-modifying agents, and the like. The composition may be formulated for any appropriate mode of administration, such as oral, parenteral, transdermal, and the like. For each such mode of administration, suitable pharmaceutically acceptable additives are known in the art.

In embodiments the compositions comprise the SR16388 and inhibitor copackaged or coformulated together, and optionally with one or more of the additional, different anti-cancer medicaments. For example, the combinations may be coformulated, particularly in unit dosage form, or unit dosage forms of each of the SR16388 and inhibitor may be copackaged in a multipack adapted for sequential use, such as blisterpack, comprising sheets of unit dosage forms.

Determination of IC₅₀ values (i.e., the concentration of drug required to kill 50% of cells in exponentially growing cultures after a 24 h exposure to the drug) can be accomplished using any appropriate technique, such as the technique that follows (adapted from Liebmann et al., Br. J. Cancer (1993), 68, 1104-1109). The target cells are maintained in RPMI 1640 medium supplemented with 10% fetal bovine serum (FBS) and antibiotics, or may be grown in Dulbecco's modified minimal essential medium (DMEM) supplemented with 20% FBS and antibiotics. A number of 100 mm petri dishes are plated with 5×10⁵ cells. Exponentially growing human tumor cell lines are exposed for 24 h to drug formulated in Cremophor EL. Cells are counted after the exposure and the results plotted to determine IC₅₀.

Unless otherwise indicated, the disclosure is not limited to specific procedures, materials, or the like, as such may vary. It is also to be understood that the terminology used herein is for the purpose of describing particular embodiments only and is not intended to be limiting.

As used in the specification and the appended claims, the singular forms “a,” “an,” and the include plural referents unless the context clearly dictates otherwise. Thus, for example, reference to “a solvent” includes not only a single solvent but also a combination or mixture of two or more different solvents.

The invention encompasses all combinations of recited particular and preferred embodiments. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

### EXAMPLES

### General Procedures

Cells were treated with compounds alone or in combination for three days and CellTiter-Glo® Luminescent Cell Viability Assay (Promega Corp.) was used as the endpoint to measure cell viability.

Cells were cultured in DMEM with 10% fetal bovine serum and used between passage 2 and passage 20. Cells were harvested on Day-1 (FIG. 1A) and re-supspended to the corresponding seeding density and seeded with Wellmate (ThermoFisher) to 384 plates (BD or GBO).

SR16388 was synthesized in house. Paclitaxel and Vincristine were purchased from commercial sources (Sigma, AK Scientific). 40 mM Compound stocks in 100% DMSO were made. Master plates were made with Janus (PerkinElmer), then further diluted and added to the cell plates with Matrix Platemate (Thermo) on Day 0.

CellTiter-Glo® Luminescent Cell Viability Assay (Promega Corp.) was used to measure ATP as an indicator of viable cells on Day 3 using Envision (PerkinElmer) and back up reader Analyst HT (MDS) and Gen5 (BioTek).

Single point, 5×4 checkque board platemap was used in the initial screening.

Relative Luminescent Units (RLU) were plotted against corresponding drug concentrations and fitted with standard four parameter sigmoidal curve with custom coded R program or Calcusyn (Biosoft, Cambridge, UK).

General Observations

The best synergistic interaction was mostly observed with the ratio of SR16388:Paclitaxel=83:17 or 67:33. With more paclitaxel in the mixture, antagonist interactions were observed. Confirmed the synergistic anti-proliferation effect of SR16388 in combination with four mitotic inhibitors: Paclitaxel, Vincristine, Docetaxel or Vinorelbine.

### Example 1

Combination of SR16388 with 30 compounds tested on a panel of 48 to 60 cell lines. Synergistic effects were identified by: 1) pattern recognition of the dose response curve shifts of the reference compound; and 2) algorisms based on Lowes' additivity and statistical identification of outliners in HTS.

Synergistic anti-proliferation was observed with SR16388 in combination with mitotic inhibitors (microtubulin inhibitors) only, particularly Paclitaxel and Vincristine and Docetaxel, among 30 cancer therapeutic agents examined.

TABLE 1
Synergistic combination identified by pattern recognition

| | | | | | | % |
|---|---|---|---|---|---|---|
| Compound | source | no data | synergy | no effect | total | synergy |
| Paclitaxel | 5x4-T1 | 2 | 40 | 18 | 60 | 69 |
| Vincristine | 5x4-T1 | 18 | 23 | 19 | 60 | 55 |
| SN38 | 5x4-T1 | 2 | 13 | 45 | 60 | 22 |
| Dasatinib | 5x4-T1 | 2 | 8 | 50 | 60 | 14 |
| Docetaxel | 5x4-T1 | 2 | 7 | 51 | 60 | 12 |
| Romidepsin | 5x4-T1 | 2 | 6 | 52 | 60 | 10 |
| Actinomycin | 5x4-T1 | 18 | 3 | 39 | 60 | 7 |
| Tamoxifen | 5x4-T1 | 18 | 3 | 39 | 60 | 7 |
| Rapamycin | 5x4-T1 | 2 | 4 | 54 | 60 | 7 |
| Lapatinib | 5x4-T1 | 2 | 3 | 55 | 60 | 5 |
| Sunitinib | 5x4-T1 | 2 | 2 | 56 | 60 | 3 |
| Imatinib | 5x4-T1 | 18 | 1 | 41 | 60 | 2 |
| Carboplatin | 5x4-T1 | 18 | 1 | 41 | 60 | 2 |
| Everolimus | 5x4-T1 | 2 | 0 | 58 | 60 | 0 |
| Nilotinib | 5x4-T1 | 18 | 0 | 42 | 60 | 0 |
| Capecitabine | 5x4-T1 | 2 | 0 | 58 | 60 | 0 |
| Celecoxib | 5x4-T1 | 18 | 0 | 42 | 60 | 0 |
| Cladribine | 5x4-T1 | 18 | 0 | 42 | 60 | 0 |
| Clofarabine | 5x4-T1 | 2 | 0 | 58 | 60 | 0 |
| Cytarabine | 5x4-T1 | 18 | 0 | 42 | 60 | 0 |
| Floxuridine | 5x4-T1 | 18 | 0 | 42 | 60 | 0 |
| FluroaraA | 5x4-T1 | 18 | 0 | 42 | 60 | 0 |
| Gemcitabine | 5x4-T1 | 2 | 0 | 58 | 60 | 0 |
| Letrozole | 5x4-T1 | 18 | 0 | 42 | 60 | 0 |
| Nelarabine | 5x4-T1 | 2 | 0 | 58 | 60 | 0 |
| Temozolomide | 5x4-T1 | 18 | 0 | 42 | 60 | 0 |
| Thiotepa | 5x4-T1 | 18 | 0 | 42 | 60 | 0 |
| Tretinoin | 5x4-T1 | 18 | 0 | 42 | 60 | 0 |
| Vorinostat | 5x4-T1 | 2 | 0 | 58 | 60 | 0 |

Synergistic Combinations Identified

1) For each specific combination (e.g. compound A at concentration a1 combined with compound B at concentration b1 in cell line X), the predicted effect of compound B in combination was calculated by multiplying the available signal window after the effect of compound A with the percent inhibition of compound B alone at that concentration (Eb1−es=(Ecrtl−Ea1)*% inhibit b1);

2) The observed effect of compound B was calculated by subtracting the effect of compound A from the combination effect (Eb1−ob=Ea1b1−Ea1);

3) The difference between the predicated effect and the observed effects was compared to the standard deviation (SD) of compound A at concentrational to obtain the Z score of the combination (Za1b1x=(Eb1−es−Eb1−ob)/SDcrtl;

4) Standard deviation (SD) of the Z score was calculated using the Z scores of non-effective combinations (the combination with lowest concentration of compound A and compound B);

5) A “combination score” was assigned to each specific combination (Ca1b1x): C=1 if Za1b1x>3*SD and C=0 if Za1b1x≦3*SD score);

6) Hit rate of each combination was calculated by comparing the sum of the combination scores of all 20 (5×4) dose combinations with all cell lines to the total number of data points of that specific combination;

7) Hit rate were ranked from high to low to identify potential synergistic combinations.

TABLE 2
Synergism hit rate for various drug combinations

| M Drug | % hit/combo |
|---|---|
| Paclitaxel | 2.25 |
| Vincristine | 2.08 |
| Actinomycin | 1.67 |
| Sunitinib | 1.25 |
| Capecitabine | 1.08 |
| Dasatinib | 1.08 |
| Imatinib | 0.92 |
| Gemcitabine | 0.83 |
| Rapamycin | 0.83 |
| Vorinostat | 0.83 |
| Carboplatin | 0.83 |
| Tamoxifen | 0.83 |
| Thiotepa | 0.83 |
| Romidepsin | 0.75 |
| Temozolomide | 0.75 |
| Lapatinib | 0.67 |
| Celecoxib | 0.67 |
| Cladribine | 0.67 |
| Floxuridine | 0.67 |
| FluroaraA | 0.67 |
| Docetaxel | 0.58 |
| SN38 | 0.58 |
| Tretinoin | 0.58 |
| Clofarabine | 0.50 |
| Everolimus | 0.42 |
| Letrozole | 0.42 |
| Nelarabine | 0.33 |
| Cytarabine | 0.33 |
| Nilotinib | 0.33 |

### Example 2

The example provides data from 11×7 experiments to measure synergism hit rate in combinations of SR16388 with Paclitaxel, Docetaxel, Vincristine, or Vinblastine. Within the same type of cancer cells, some cell lines have more synergistic hits than others. Melanoma and breast cancer cell lines had the highest hit rate.

TABLE 3
Synergistic hit rate across various cancer cell lines

% Hit rate¹

| Cell type | Cell line | D | P | V1 | V2 |
|---|---|---|---|---|---|
| NSCLC | NCIH460 | 31 | 35 | 16 | 19 |
| Melanoma | SKMEL28 | 31 | 35 | 8 | 22 |
| Breast Cancer | MCF7 | 19 | 31 | 17 | 25 |
| Melanoma | UACC257 | 18 | 35 | 6 | 25 |
| CNS Cancer | SNB19 | 18 | 27 | 12 | 27 |
| Colon Cancer | COLO205 | 35 | 17 | 12 | 14 |
| Melanoma | UACC62 | 16 | 25 | 10 | 23 |
| Breast Cancer | HS578T | 22 | 23 | 8 | 17 |
| Melanoma | SKMEL2 | 12 | 29 | 3 | 25 |
| Leukemia | HL60 | 21 | 25 | 16 | 5 |
| CNS Cancer | SF268 | 18 | 22 | 6 | 18 |
| Breast Cancer | T47D | 19 | 19 | 9 | 16 |
| NSCLC | NCIH226 | 19 | 27 | 8 | 8 |
| Renal Cancer | CAKI1 | 16 | 22 | 12 | 13 |
| Colon Cancer | HT29 | 21 | 22 | 8 | 10 |
| Renal Cancer | 7860 | 10 | 22 | 10 | 18 |
| Ovarian Cancer | SKOV3 | 12 | 18 | 8 | 19 |
| Leukemia | MOLT4 | 13 | 21 | 8 | 16 |
| NSCLC | NCIH322M | 14 | 17 | 3 | 18 |
| CNS Cancer | SF539 | 13 | 19 | 8 | 12 |
| Breast Cancer | MDAMB231 | 10 | 21 | 1 | 17 |
| Colon Cancer | HCT15 | 12 | 14 | 10 | 9 |
| Colon Cancer | HCT116 | 18 | 13 | 6 | 6 |
| Melanoma | SKMEL5 | 10 | 12 | 5 | 14 |
| Renal Cancer | TK10 | 10 | 13 | 4 | 14 |
| CNS Cancer | SF295 | 8 | 8 | 0 | 21 |
| Leukemia | CCRFCEM | 12 | 14 | 3 | 8 |
| Melanoma | MDAMB435 | 14 | 12 | 4 | 6 |
| Renal Cancer | SN12C | 8 | 13 | 1 | 14 |
| Melanoma | MALME | 6 | 14 | 0 | 14 |
| Ovarian Cancer | OVCAR8 | 6 | 13 | 5 | 10 |
| NSCLC | NCIH522 | 8 | 10 | 4 | 10 |
| CNS Cancer | SNB75 | 5 | 13 | 3 | 9 |
| Colon Cancer | SW620 | 8 | 9 | 8 | 5 |
| NSCLC | A549 | 4 | 16 | 0 | 10 |
| Renal Cancer | A498 | 4 | 10 | 4 | 12 |
| Colon Cancer | HCC2998 | 6 | 12 | 6 | 4 |
| NSCLC | HOP92 | 4 | 10 | 6 | 8 |
| Ovarian Cancer | IGROV1 | 3 | 18 | 0 | 8 |
| Renal Cancer | ACHN | 4 | 14 | 3 | 8 |
| Melanoma | LOXIMVI | 8 | 13 | 1 | 5 |
| Renal Cancer | U031 | 4 | 9 | 3 | 12 |
| CNS Cancer | U251 | 12 | 12 | 0 | 4 |
| Colon Cancer | KM12 | 6 | 10 | 1 | 6 |
| Breast Cancer | MDAMB468 | 6 | 8 | 3 | 5 |
| Leukemia | K562 | 0 | 12 | 3 | 8 |
| Ovarian Cancer | OVCAR3 | 8 | 8 | 4 | 3 |
| Breast Cancer | BT549 | 5 | 3 | 1 | 9 |
| NSCLC | NCIH23 | 5 | 4 | 4 | 4 |
| Ovarian Cancer | OVCAR5 | 1 | 6 | 0 | 1 |
| Leukemia | RPMI8226 | 1 | 5 | 0 | 0 |
| Ovarian Cancer | ADRRES | 3 | 1 | 0 | 1 |
| Prostate Cancer | DU145 | 1 | 3 | 0 | 1 |
| Prostate Cancer | PCS | 3 | 0 | 1 | 1 |
| NSCLC | HOP62 | 1 | 1 | 0 | 0 |
| NSCLC | EKVX | 0 | 0 | 1 | 1 |
| Leukemia | SR | 0 | 1 | 0 | 0 |

¹D = Docetaxel; P = Paclitaxel, V1 = Vinblastine; V2 = Vincristine

SR16388 was found to have synergistic effect with all tested mitosis inihibitors, with following relative frequency: Paclitaxel>Docetaxel>Vincristine>Vinblastine.

TABLE 4
synergistic combination identified by algorism

| Drug | % Hit Rate |
|---|---|
| Paclitaxel | 14.88 |
| Vincristine | 10.91 |
| Docetaxel | 10.69 |
| Vinblastine | 4.94 |

Melanoma cell lines were found to have the highest synergistic hit rate among 9 different cancer cell types, followed by breast cancer cell lines.

TABLE 5
Frequency of synergism across various cancers

| Type | Cell line # | % Hit/cell line |
|---|---|---|
| Melanoma | 8 | 14.45 |
| Breast Cancer | 6 | 13.15 |
| CNS Cancer | 6 | 12.28 |
| Colon Cancer | 7 | 11.13 |
| Renal Cancer | 7 | 10.25 |
| NSCLC | 9 | 9.13 |
| Leukemia | 6 | 7.90 |
| Ovarian Cancer | 6 | 6.55 |
| Prostate Cancer | 2 | 1.30 |

Actinomycin, an anti-cancer agent that is anti-metabolitic rather than anti-mitotic, was found to have a very low hit rate of 1.89% when tested with SR16388.

Important Observations

The reason that the synergy of SR16388 with microtubulin inhibitors paclitxel or vincristine was particularly surprising is that we discovered it as a result of performing a non-bias screen with 30 different standard chemotherapeutic agents representing many different mechanisms of action and only microtubulin inhibitors, and particularly paclitxel and vincristine, demonstrated synergy across a broad panel (over 20) cancer cell lines of different tissues of origin.

The synergy between SR16388 and microtubulin inhibitors, particularly paclitxel or vincristine was unexpected and could not have been predicted from any mechanistic information on this compound. The lack of synergy with all the other agents, meaning the specificity of this effect, was also surprising.

The disclosed interaction between SR16388 and microtubulin inhibitors, particularly paclitxel or vincristine, was defined as synergy by art-accepted analysis algorithms, and is not simple additive activity.

Our prior vivo xenograft study demonstrated the feasibilty that SR16388 could be combined with standard therapies, such as paclitexel, without limitations of (overlaping) toxicities. It did not suggest or demonstrate synergy because the doses of the individual agents were too potent to allow a large enough window to see synergistic effects, nor did or could the study address the specificity of this effect relative to other chemotherapeutic agents.

The invention provides an unexpected finding of remarkable selective synergy between SR16388 and microtubulin inhibitors, particularly paclitxel or vincristine in broad cancer types. The invention indicates that SR16388 can be added to chemothreapeutic regimens to produce clinical benefits greater than those expected by from the simple additive activity of the single agents. Since, SR16388 is well tolerated and does not have ovelapping toxicities with microtubulin inhibitors (demonstrated by the xenograft study and other safety data) this combination is clinically valuable. Prostate, breast and lung cancers are preferred targets since paclitaxel is a chemotherapy currently used in these cancer types.

1. A method for inhibiting tumor growth in a mammal comprising administering to the mammal a synergistic combination of SR16388 and a microtubulin inhibitor, wherein the combination more than additively inhibits growth of ovarian, colon, breast, prostate, lung, or myeloma tumor cells.

2. The method of claim 1 wherein the combination is administered in a single composition.

3. The method of claim 1 wherein one or both of the SR16388 and inhibitor is administered in an amount subtherapeutic if administered alone.

4. The method of claim 1, wherein one or both of the SR16388 and inhibitor is administered in an amount less than its IC₅₀ for said cells.

5. The method of claim 1, wherein the combination is administered in an amount less than its IC₅₀ for said cells.

6. The method of claim 1, wherein the inhibitor is selected from the group consisting of paclitaxel, docetaxel, vinblastine, vinorelbine, and vincristine, or combinations thereof.

7. The method of claim 1, wherein the inhibitor is paclitaxel or vincristine.

8. The method of claim 1, wherein the SR16388:inhibitor ratio is between 90:10 and 60:40.

9. The method of claim 1, wherein the combination is synergistic for a plurality of cell lines selected from NCIH460, SKMEL28, MCF7, UACC257, SNB19, COLO205, UACC62, HS578T, SKMEL2, HL60, SF268, T47D, NCIH226, CAKI1, HT29, 786O, SKOV3, MOLT4, NCIH322M, SF539, MDAMB231, HCT15, HCT116, SKMEL5, TK10, SF295, CCRFCEM, MDAMB435, SN12C, MALME, OVCAR8, NCIH522, SNB75, SW620, A549, A498, HCC2998, HOP92, IGROV1, ACHN, LOXIMVI, UO31, U251, KM12, MDAMB468, K562, OVCAR3, BT549, NCIH23, OVCAR5, RPMI8226, ADRRES, DU145, PC3, HOP62, EKVX, and SR.

10. The method of claim 1, wherein the combination exhibits greater synergy compared with a composition comprising SR16388 and an anti-cancer drug selected from SN38, Dasatinib, Romidepsin, Actinomycin, Tamoxifen, Rapamycin, Lapatinib, Sunitinib, Imatinib, Carboplatin, Everolimus, Nilotinib, Azacytidine, Capecitabine, Celecoxib, Cladribine, Clofarabine, Cytarabine, Floxuridine, FluroaraA, Gemcitabine, Letrozole, Nelarabine, Temozolomide, Thiotepa, Tretinoin, and Vorinostat.

11. The method of claim 1, wherein the microtubulin inhibitor is paclitaxel, the combination is administered in a single composition, and the SR16388:inhibitor ratio is between 85:15 and 65:35.

12. The method of claim 1, wherein the inhibitor is paclitaxel or vincristine, the combination is administered in a single composition, and the inhibitor is administered in an amount subtherapeutic if administered alone.

13. The method of claim 1, wherein the inhibitor is paclitaxel or vincristine, the combination is administered in a single composition, and the tumor is an ovarian, colon, breast, prostate, lung or myeloma tumor.

14. The method of claim 1, wherein the method further comprises the step of detecting a resultant inhibition of tumor growth.

15. A pharmaceutical composition comprising a synergistic composition, with respect to tumor growth inhibition, of SR16388 and a microtubulin inhibitor, wherein the composition provides a synergistic inhibition of ovarian, colon, breast, prostate, lung or myeloma tumor growth.

16. The method of claim 1, wherein:
the inhibitor is paclitaxel or vincristin,
the SR16388:inhibitor ratio is between 90:10 and 60:40,
the combination is synergistic for a plurality of cell lines selected from NCIH460, SKMEL28, MCF7, UACC257, SNB19, COLO205, UACC62, HS578T, SKMEL2, HL60, SF268, T47D, NCIH226, CAKI1, HT29, 786O, SKOV3, MOLT4, NCIH322M, SF539, MDAMB231, HCT15, HCT116, SKMEL5, TK10, SF295, CCRFCEM, MDAMB435, SN12C, MALME, OVCAR8, NCIH522, SNB75, SW620, A549, A498, HCC2998, HOP92, IGROV1, ACHN, LOXIMVI, UO31, U251, KM12, MDAMB468, K562, OVCAR3, BT549, NCIH23, OVCAR5, RPMI8226, ADRRES, DU145, PC3, HOP62, EKVX, and SR, and
the tumor is an ovarian, colon, breast, prostate, lung or myeloma tumor.

17. The method of claim 1, wherein:
the inhibitor is paclitaxel or vincristin,
the SR16388:inhibitor ratio is between 90:10 and 60:40,
the combination is synergistic for a plurality of cell lines selected from NCIH460, SKMEL28, MCF7, UACC257, SNB19, COLO205, UACC62, HS578T, SKMEL2, HL60, SF268, T47D, NCIH226, CAKI1, HT29, 786O, SKOV3, MOLT4, NCIH322M, SF539, MDAMB231, HCT15, HCT116, SKMEL5, TK10, SF295, CCRFCEM, MDAMB435, SN12C, MALME, OVCAR8, NCIH522, SNB75, SW620, A549, A498, HCC2998, HOP92, IGROV1, ACHN, LOXIMVI, UO31, U251, KM12, MDAMB468, K562, OVCAR3, BT549, NCIH23, OVCAR5, RPMI8226, ADRRES, DU145, PC3, HOP62, EKVX, and SR,
the combination is administered in a single composition, and
the tumor is an ovarian, colon, breast, prostate, lung or myeloma tumor.

18. The composition of claim 15, wherein the inhibitor is paclitaxel or vincristine.

19. The composition of claim 15, wherein the inhibitor is paclitaxel or vincristine, and the SR16388:inhibitor ratio is between 90:10 and 60:40.

20. The composition of claim 15, wherein the inhibitor is paclitaxel or vincristine, the SR16388:inhibitor ratio is between 90:10 and 60:40, and the inhibitor is an amount subtherapeutic if administered alone.
